(19) United States
(12) Patent Application Publication
(10) Pub. No.: US 2022/0400940 A1
(43) Pub. Date: Dec. 22, 2022

(54) METHOD FOR OBTAINING IMAGES FOR ASSESSING ORAL HEALTH USING A MOBILE DEVICE CAMERA AND A MIRROR

(71) Applicant: Adent ApS, Copenhagen K (DK)

(72) Inventors: Richard BUNDSGAARD, Copenhagen K (DK); Thøger BUNDSGAARD, Copenhagen K (DK)

(73) Assignee: Adent ApS, Copenhagen K (DK)

(21) Appl. No.: 17/765,792

(22) PCT Filed: Oct. 1, 2020
(86) PCT No.: PCT/EP2020/077542

§ 371 Date: Mar. 31, 2022

(30) Foreign Application Priority Data
Oct. 4, 2019 (DK) PA 2019 70622

(51) Int. Cl. A61B 1/24; A61B 5/00; G16H 80/00

(52) U.S. Cl. CPC A61B 1/24; A61B 5/6898; A61B 5/0079; A61B 5/0013; A61B 5/742; A61B 5/7485; A61B 5/7465; A61B 5/4542; G16H 80/00

(57) ABSTRACT

A method for obtaining images for assessing oral health using a mobile device (103) having a screen (205) on one side of a housing of the mobile device (103) and a camera (207) on the opposite side of the housing (103), comprising: —on the mobile device (103) performing a dialogue with a user (101) of the mobile device (103), —determining an area of the user's oral cavity with symptoms based on the dialogue, —instructing the user (101) to position him-/herself in front of a minor (102) for capturing an image of the area of the user's oral cavity with symptoms, —capturing at least one image of the area of the user's oral cavity with symptoms with the camera (207) as instructed, and—either processing the at least one image locally on the mobile device (103), or—transmitting the at least one image or a representation thereof to a remote server (409).

### TECHNICAL FIELD

The present disclosure relates generally to the field of oral health, and more particularly to a method for obtaining images for oral health assessment using a mobile device camera and a mirror.

### BACKGROUND

Oral health assessment is crucial for early intervention against and preventive treatment of oral health problems. However, many people do not receive proper oral health assessment, for several reasons. For example, they may live in rural areas far from a dental clinic and may, hence, not have access to oral health assessment, or they may not have the economic means to consult with a dental professional, let alone to pay for a potential treatment. Further, many people may be disinclined to spend time and money on an initial oral health assessment or on regular dental checkups if they are not experiencing any apparent oral health problem, despite the fact that, sometimes, people may unknowingly have symptoms of compromised oral health or unknowingly be at risk of developing an oral health problem. In other cases, people may be experiencing a certain oral health problem or symptoms of an oral disease but may decide to wait before consulting with a professional, hoping that the problem or symptoms will go away. All these example scenarios are problematic since early and proper oral health assessment allows for early intervention and preventive treatment, thus preventing or reducing the occurrence or progress of many oral problems and diseases.

Systems for remote oral health assessment, often referred to as teledentistry, exist. These systems typically provide real-time and offline dental care such as diagnosis, treatment planning, consultation, and follow-up through electronic transmission of clinical data among patients and dental professionals, e.g. using smartphones or other mobile devices. Some known remote oral health assessment systems involve transmission of e.g. dental images from the patient. For example, the patient may use the front camera of his/her smartphone for capturing a dental image and may transmit the captured image via e.g. a web-based application on the smartphone for remote clinical assessment.

However, the obtaining of proper images (images of clinical quality) of an area of the oral cavity, e.g. dental images, using a smartphone camera is challenging. If the smartphone camera is held too far from the teeth, the image may not have sufficient detail to be of clinical value. If the smartphone camera is held too close to the teeth, the image may be blurry and optically distorted, and the smartphone may block for sufficient light access. Further, it may be difficult or even impossible for a patient to capture proper images of e.g. the upper or lower jaw or wisdom teeth at the back of the oral cavity all by him-/herself, and many may feel embarrassed to ask a family member or friend for help. Often, in order to obtain an image with the right angle of view, the patient will have to position the smartphone camera in a way that partly or entirely hinders him/her from viewing the smartphone screen. This further complicates the obtaining of a proper image since the patient will not be able to view the detected image represented in real-time on the smartphone screen and may therefore not easily check and adjust e.g. the focus, lighting, level of detail, zoom factor, etc. prior to capturing and saving the image.

Accordingly, there is a need for technologies that provide users with easy and cheap access to oral health assessment and with solutions for conveniently and independently obtaining high-quality oral cavity images for the assessment.

### SUMMARY

It is an object to overcome or at least reduce the problems mentioned above.

The foregoing and other objects are achieved by the features of the independent claims. Further implementation forms are apparent from the dependent claims, the description, and the figures.

According to a first aspect, there is provided a method for obtaining images for assessing oral health using a mobile device having a screen on one side of a housing of the mobile device and a camera provided on the opposite side of the housing of the mobile device, the method comprising:
on the mobile device performing a dialogue with a user of the mobile device,
the dialogue comprising prompting one or more questions to the user via a user interface of the mobile device and recording answers to the questions from the user via the user interface of the mobile device,
determining an area with symptoms of said user's oral cavity based on said user's answers to said questions,
instructing the user to position him-/herself in front of a mirror for capturing an image of the area of the user's oral cavity with symptoms,
instructing the user to rotate the mobile device such that the screen faces the mirror and the camera faces the user, with the camera being directed towards the area of the user's oral cavity with symptoms for capturing an image of the area of the user's oral cavity with symptoms,
capturing at least one image of the area of the user's oral cavity with symptoms with the camera of the mobile device as instructed, and
either processing the at least one captured image locally on the mobile device for assessment on the mobile device of the user's oral health based on the at least one captured image, or
transmitting the at least one captured image or a representation thereof from the mobile device to a remote server for remote assessment of the user's oral health based on the at least one captured image.

The effect hereof is that the user is able to obtain one or more images of an area of his/her oral cavity without the need of dedicated dental equipment (such as a dental mirror) or any tools that are not normally present in an average household, using only a mobile phone camera, for assessment of his/her oral health based on the images. With the method it also becomes possible to take an adequate image of an area of a user's oral cavity with symptoms without the need for taking multiple images or using a timer or other alert conventionally used for taking self-portraits.

The user is able to view real-time images detected with the camera and displayed on the screen reflected in the mirror. Thus, the user may view his/her own oral cavity in real-time in the mirror and may use this as information input for positioning/guiding the camera in order to capture a desired image. The user may, for example, check and adjust image properties such as e.g. the focus, lighting, level of detail, or zoom factor of an image prior to capturing and saving the image on the mobile device and/or transferring the image to a remote server.

In a possible implementation form of the first aspect, the method comprises:
instructing the user to rotate the camera to landscape position and
capturing at least one image in landscape mode of the area of the user's oral cavity with the camera of the mobile device.

In a possible implementation form of the first aspect, the method comprises instructing the user to position him-/herself such that the mirror is at a given distance from the user's oral cavity.

In a possible implementation form of the first aspect, the method comprises capturing with the camera of the mobile device an image of at least a portion of at least one of a tooth, a lip, or gums.

In a possible implementation form of the first aspect, the mobile device is a smartphone or a tablet.

In a possible implementation form of the first aspect, the method comprises displaying the image captured by the camera in real-time on the screen of the mobile device.

In a possible implementation form of the first aspect, the method comprises instructing the user to view the reflection of the area of the user's oral cavity displayed on the screen of the mobile device in the mirror before/when capturing the image of the area of the user's oral cavity.

In a possible implementation form of the first aspect, the sequence of questions comprises questions posed in the form of at least one of free text, spoken text, a checklist, a slider bar, or a visual representation.

In a possible implementation form of the first aspect, the sequence of questions to the user comprises questions relating to at least one of pain, history of occurrence of symptoms, symptom triggers, or urgency.

In a possible implementation form of the first aspect, the symptoms are symptoms of at least one of gingivitis, periodontitis, dental caries, abrasion of tooth, bruxism, cold sore, erosion of teeth, fluorosis, herpes labialis, herpes zoster, or herpes infection.

In a possible implementation form of the first aspect, the method comprises automatically checking with the mobile device the image quality of the captured image or of a representation thereof and instructing the user to capture another image of the area of the user's oral cavity if the image quality is below a threshold for image quality.

In a possible implementation form of the first aspect, the method comprises automatically checking on the remotely located server the image quality of the captured image or of a representation thereof and instructing the user to capture another image of the area of the user's oral cavity if the image quality is below a threshold for image quality.

In a possible implementation form of the first aspect, the method comprises guiding the user to capture an image of the area of the user's oral cavity in a particular view.

In a possible implementation form of the first aspect, the particular view may be a closed mouth view, bite view, lower arch view, upper arch view, lower lip view, upper lip view, closed bite anterior view, open bite anterior view, closed bite buccal view, open bite buccal view, roof of mouth view, floor of mouth view, side of mouth view, or full frontal view.

In a possible implementation form of the first aspect, the method comprises, prior to transmitting the captured image to the remote server, annotating the image as displaying a closed mouth view, bite view, bottom arch view, lower arch view, upper arch view, lower lip view, upper lip view, closed bite anterior view, open bite anterior view, closed bite buccal view, open bite buccal view, roof of mouth view, floor of mouth view, side of mouth view, or full frontal view.

In a possible implementation form of the first aspect, the method comprises performing the remote assessment by a human expert.

In a possible implementation form of the first aspect, the method comprises performing the remote assessment using artificial intelligence.

In a possible implementation form of the first aspect, the method comprises using the captured image as input for statistical algorithms and machine learning technologies.

In a possible implementation form of the first aspect, the method comprises transmitting the captured image to the remote server for determining whether the user is a candidate for a dental treatment based on the captured image and informing the user on the mobile device that the user is or is not a candidate for a dental treatment.

In a possible implementation form of the first aspect, the method comprises determining with the mobile device the area of the user's oral cavity with symptoms based on the user's answers to the questions.

In a possible implementation form of the first aspect, the method comprises determining remotely from the mobile device the area of the user's oral cavity with symptoms based on the user's answers to the questions, preferably by a remotely located human expert and/or by artificial intelligence on a remotely located server.

In a possible implementation form of the first aspect, the method comprises assessing the user's oral health based on the at least one captured image.

According to a second aspect, there is provided a machine-readable storage medium encoded thereon with a computer program product operable to cause a processor to perform operations according to the methods of any one of the possible implementation forms of the first aspect.

According to a third aspect, there is provided system for assessing oral health of a user, the system comprising:

a mobile device comprising a screen on one side of a housing of the mobile device and a camera provided on the opposite side of the housing of the mobile device, the camera configured to obtain an image of the user's oral cavity;

a machine-readable storage medium comprising a program product; and

one or more processors operable to execute the program product and perform operations according to the methods of any one of the possible implementation forms of the first aspect.

These and other aspects will be apparent from and the embodiment(s) described below.

### BRIEF DESCRIPTION OF THE DRAWINGS

In the following detailed portion of the present disclosure, the aspects, embodiments, and implementations will be explained in more detail with reference to the example embodiments shown in the drawings, in which:

FIG. 1 is a diagrammatic representation according to an embodiment, showing a user, a mirror, the user's reflection in the mirror, a mobile device, and an image of an area of the user's oral cavity, which image is displayed on the main screen of the mobile device;

FIG. 2a is a front view of a mobile device according to an embodiment showing a main screen and a front camera;

FIG. 2b is a rear view of the mobile device of FIG. 2a, showing a main camera and a flashlight;

FIG. 3 is a diagrammatic representation of the user, mirror, and mobile device of FIG. 1, the reflections of the user and the mobile device in the mirror, and the obtaining of an image of an area of the user's oral cavity;

FIG. 4 is a diagrammatic representation of a wireless network according to an embodiment comprising a user's mobile device, a remote server, and a dental professional's computer;

FIG. 5 is a flowchart of an embodiment of a method for obtaining images for assessing a user's oral health; and

FIGS. 6 to 28 are graphical user interfaces (GUIs) displayed to the user on the main screen of the mobile device according to an embodiment, exemplifying the user's interaction with an application software executed on the mobile device.

### DETAILED DESCRIPTION

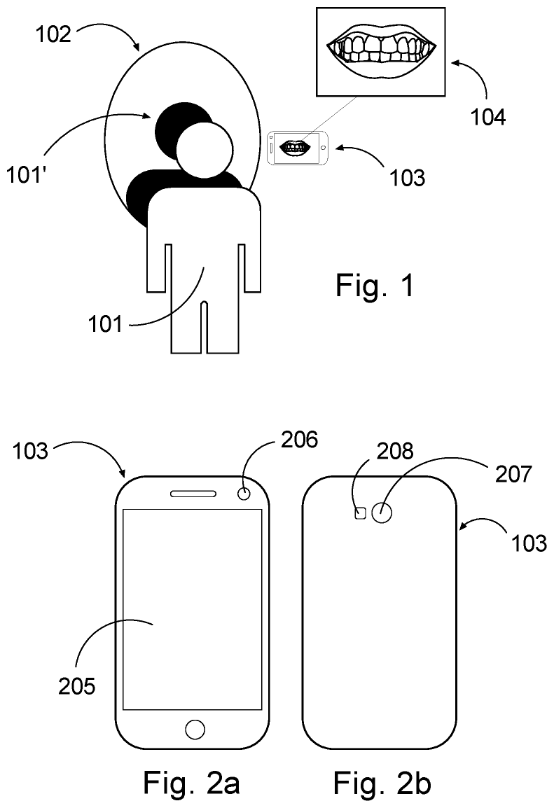

FIG. 1 is a diagrammatic representation according to an embodiment showing a user 101, a mirror 102, the user's reflection 101′ in the mirror, a mobile device 103, and an image 104 of an area of the user's oral cavity, which image 104 is displayed on the main screen of the mobile device 103.

In an embodiment, the user 101 may wish to obtain one or more images of an area of his/her oral cavity for assessment of his/her oral health. Here, “assessment” preferably refers to an assessment performed in order to determine the user's oral health condition. For example, the assessment may comprise at least one of monitoring the user's oral health condition, identifying any oral health problems, identifying any oral health risks, or determining that no symptoms of any oral health problems or diseases are present. In an embodiment, the assessment is performed automatically or semi-automatically using artificial intelligence, e.g. by using the one or more images obtained as input to statistical algorithms and machine learning methods. In an embodiment, depending on the circumstances, the assessment is performed by a human expert, e.g. a dental professional (e.g. dentist, dental hygienist, dental therapist, dental nurse, dental technician), a medical practitioner, or another health professional. The user 101 may have observed symptoms of an oral health problem or disease and may therefore wish to obtain images of an area of his/her oral cavity to have his/her oral health assessed based on the images. Or, the user 101 may not have observed any symptoms but may, nevertheless, wish to obtain images of an area of his/her oral cavity to have his/her oral health assessed. In an embodiment, the assessment may be part of a dental health treatment or follow-up plan prescribed by a dental professional.

Herein, “oral cavity” may refer to e.g. lips, hard palate, soft palate, retromolar trigone (area behind the wisdom teeth), tongue, gingiva (gums), buccal mucosa, the floor of the mouth under the tongue, and/or teeth.

Further, herein, “oral health problem” or “oral disease” may refer to at least one of Abnormal taste in mouth, Abrasion of tooth, Acid reflux, Acute necrotizing ulcerative gingivitis, Addison's disease, Alveolitis of jaw, Amalgam tattoo, Amelogenesis imperfecta, Anemia, Aphthous ulcer of mouth, Atrophy of tongue papillae, Black hairy tongue, Bleeding gums, Broken tooth injury, Bruxism (teeth grinding), Burning mouth syndrome, Cancer, Candidiasis, Cheek biting, Cheilosis, Chemical burn (mouth), Chicken pox (Varicella), Cold sore, Complete avulsion of tooth, Contusion, Crazing of enamel, Cyst, Dental caries, Dental filling lost, Dental peri-implant mucositis, Dental plaque, Dental restoration present, Dentinogenesis imperfecta, Denture stomatitis, Diastema of teeth, Early tooth exfoliation, Electrical burn, Enamel hypoplasia, Enlarged labial frenulum, Erosion of teeth, Eruption cyst, Erythema, Erythroleukoplakia of internal part of mouth, Excessive salivation, Fibroma, Fistula, Fluorosis, Fordyce's disease, Fracture of tooth, Fractured dental restoration, Geographic tongue, Gingival recession, Gingivitis, Glossopyrosis, Hemangioma, Herpes labialis, Herpes zoster, Herpes infection, Hyperplasia of gingiva, Infectious mononucleosis, Leukemia, Leukoedema, Leukoplakia, Lichen planus (mouth), Linea alba of oral mucosa, Lip biting, Lipoma, Lymphadenopathy, Malignant melanoma, Malignant tumor of major salivary gland, Malocclusion of teeth, Measles, Melanin pigmentation (mouth), Melanocytic nevus (mouth), Melanoma in situ (mouth), Mucocele of mouth, Mumps, Necrosis of the pulp, Osteoporosis, Pain in throat, Papillary hyperplasia, Papilloma, Parulis, Pemphigoid, Pemphigus, Pericoronitis, Periodontal abscess, Periodontitis, Pseudomembranous thrush, Pulpitis, Pyogenic granuloma, Rubella, Sexually transmitted infectious disease, Sialolithiasis, Sinusitis, Smokeless tobacco keratoses, Smoker's melanosis, Staining of tooth, Stomatitis, Subluxation of tooth, Syphilis, Teething syndrome, Temporomandibular joint disorder, Thermal burn, Tongue biting, Tonsillitis, Tooth absent, Tooth sensitivity to brush or floss, Tooth sensitivity to cold, Tooth sensitivity to palpation, Traumatic granuloma, Traumatic injury, Trigeminal neuralgia, Turner's Tooth, Ulcer, Verrucous carcinoma, Vitamin deficiency, Wegener's granulomatosis, White sponge nevus of mucosa, or Xerostomia.

In an embodiment, the person whose oral cavity is captured on image is the same person as the person operating the mobile device 103 for obtaining the one or more images. In another embodiment, the person operating the mobile device 103 is not the same person as the person whose oral cavity is captured on image. This may, for example, be the case when the person whose oral cavity is captured on image is a child or other person requiring help, e.g. from a parent or other family member, for capturing the one or more images of an area of his/her oral cavity.

The mobile device is a portable computing device comprising a camera and a screen. In the embodiment of FIG. 1, the mobile device 103 is a smartphone. FIG. 2a is a front view of a mobile device 103 according to an embodiment showing a main screen 205 configured to display images to the user 101 and a front camera 206. FIG. 2b is a rear view of the mobile device 103 of FIG. 2a showing a main camera 207 and a flashlight 208. In another embodiment, the mobile device 103 may be e.g. a tablet computer (tablet).

In an embodiment, the mobile device 103 is configured to execute an application software (“app”) and comprises a main camera 207 for capturing images and a main screen 205 for e.g. displaying the images, wherein the main screen 205 and the main camera 207 are provided on opposite sides of the housing of the mobile device 103. In another embodiment, the camera for capturing images may be a secondary camera and/or the screen for displaying the images may be a secondary screen provided on opposite sides of the housing of the mobile device.

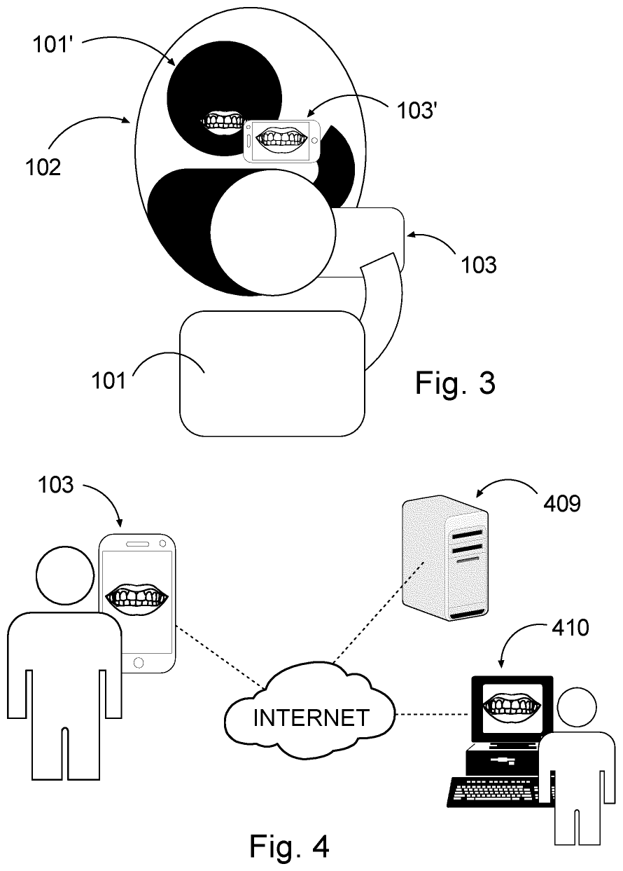

According to an embodiment, for capturing the one or more images for the oral health assessment, the user 101 is instructed by the app to position him-/herself in front of a mirror 102, as shown in FIGS. 1 and 3. FIG. 3 is a diagrammatic representation of the user 101, mirror 102, and mobile device 103 of FIG. 1, the reflections of the user 101′ and the mobile device 103′ in the mirror, and the obtaining of an image of an area of the user's oral cavity. In an embodiment, the mirror 102 is at a given distance from the user's face, preferably at a given distance from the user's oral cavity, and the app instructs the user to position him/herself accordingly. The mirror may be e.g. a wall-mounted mirror, a floor mirror, a tabletop mirror, or a handheld mirror. In the embodiment of FIG. 3, the user 101 has, upon instructions received from the app, rotated the mobile device 103 such that the main screen 205 faces the mirror 102 and the main camera 207 faces the user 101. Thus, the main screen 205, and anything displayed thereon, may be reflected in the mirror 102 and may be viewed via the mirror by the user 101.

In an embodiment, upon instruction from the app, the user 101 rotates the main camera 207 and the main screen 205 into a horizontal/landscape position for capturing an image in landscape mode, i.e. an image having more width than height, of an area of his/her oral cavity, as shown in FIG. 3.

In an embodiment, real-time images detected with the main camera 207 are displayed in real-time on the main screen 205, wherein real-time refers to the concurrent, or substantially concurrent, display of images detected by the camera 207 on the main screen 205. Thus, the user 101 may view real-time images detected with the camera 207 and displayed on the main screen 205 reflected in the mirror 102. That is, he/she may view e.g. his/her own oral cavity in real-time via the mirror 102, the main screen 205, and the main camera 207 and may use this as information input for positioning/guiding the main camera 207 in order to capture an image of an area of his/her oral cavity for oral health assessment. By being able to view images detected by the main camera 207 and represented in real-time on the main screen 205, the user may easily check and adjust e.g. the focus, lighting, level of detail, zoom factor, etc. of the images prior to capturing and saving the images on the mobile device 103 and/or transferring the captured images to a remote server 409.

The mobile device 103 is communicatively connected to a network of computer nodes, e.g. for transmitting image data and/or non-visual data from the mobile device 103 to a computer node and/or for receiving data from a computer node. FIG. 4 is a diagrammatic representation of a wireless network, such as a cellular telephone network, the internet, or a cloud-based network, comprising the mobile device 103, a remote server 409, and a dental professional's workstation 410. Image data and/or non-visual data may be transmitted from the mobile device 103 to the remote server 409 and/or the dental professional's workstation for remote assessment, processing, and/or storage of the data. The transmission of data may be synchronous, asynchronous, or a mixture of synchronous and asynchronous data transmission.

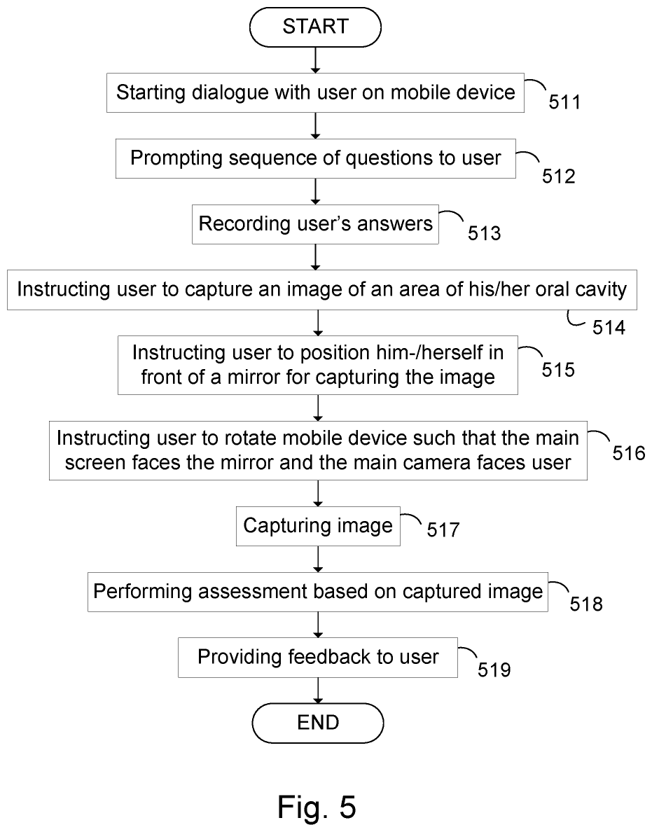

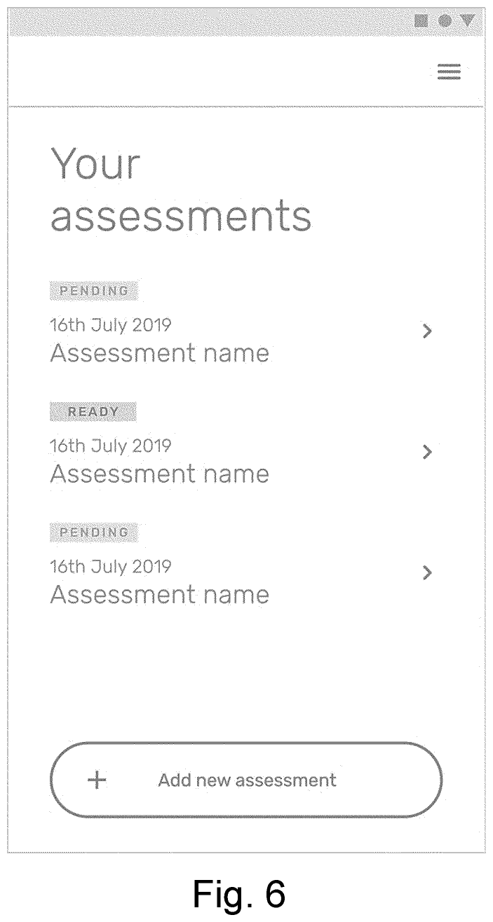

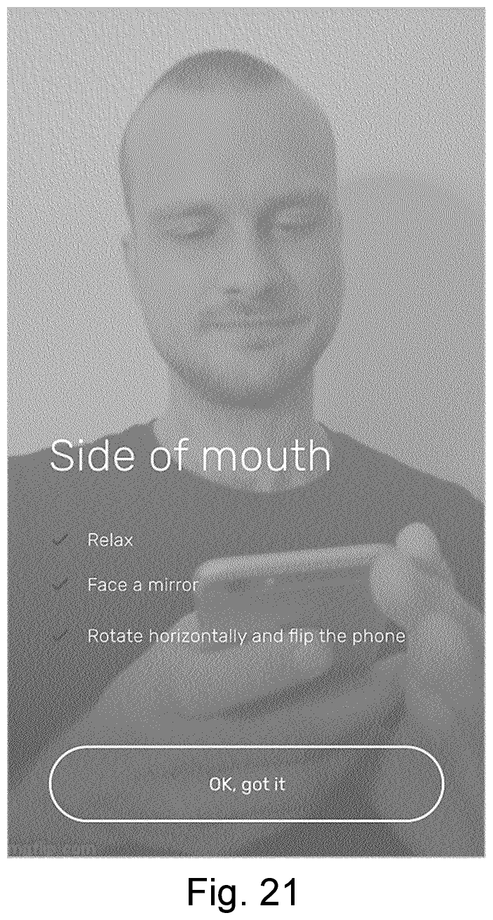

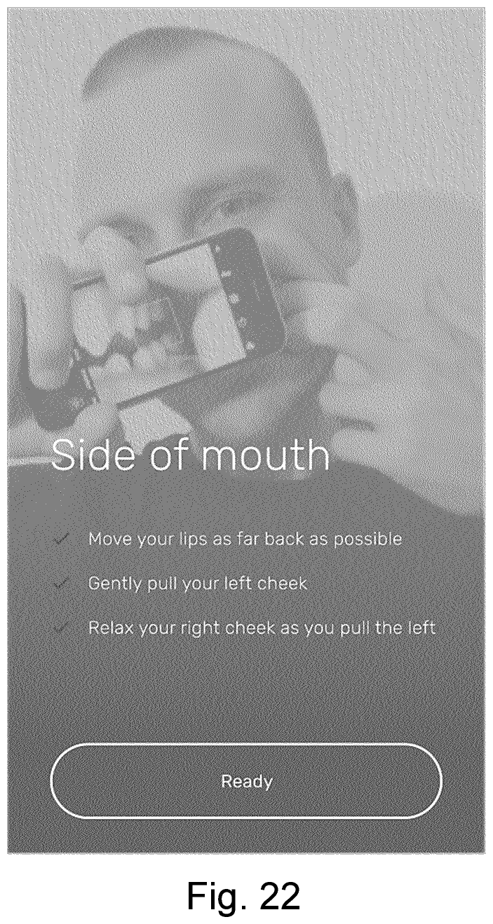

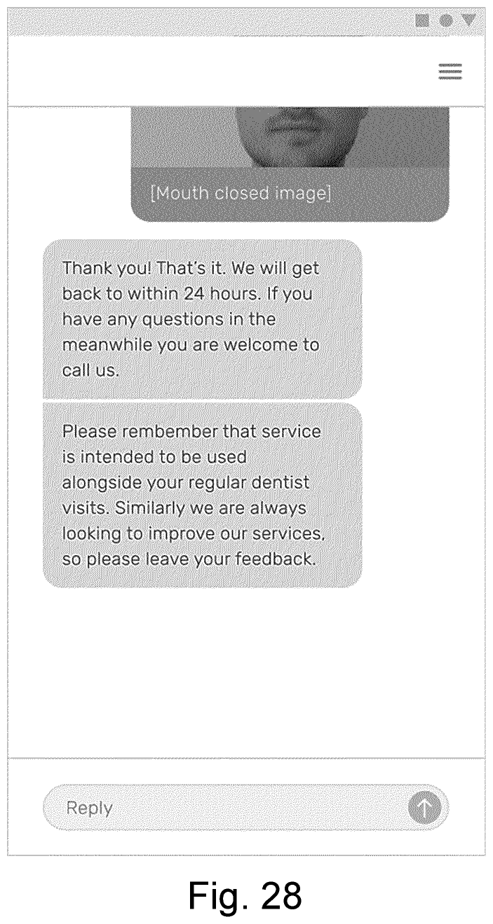

The method described herein for obtaining images for assessing oral health may at least partly be implemented in the form of an application software (“app”) which may be executed on a mobile device 103. The user 101 may interact with the application software on the mobile device 103, as described in the following with reference to the flowchart of FIG. 5 and to FIGS. 6 to 28 which are examples of graphical user interfaces (GUIs) displayed to the user 101 on the main screen 205 of the mobile device 103 during the user's interaction with the application software.

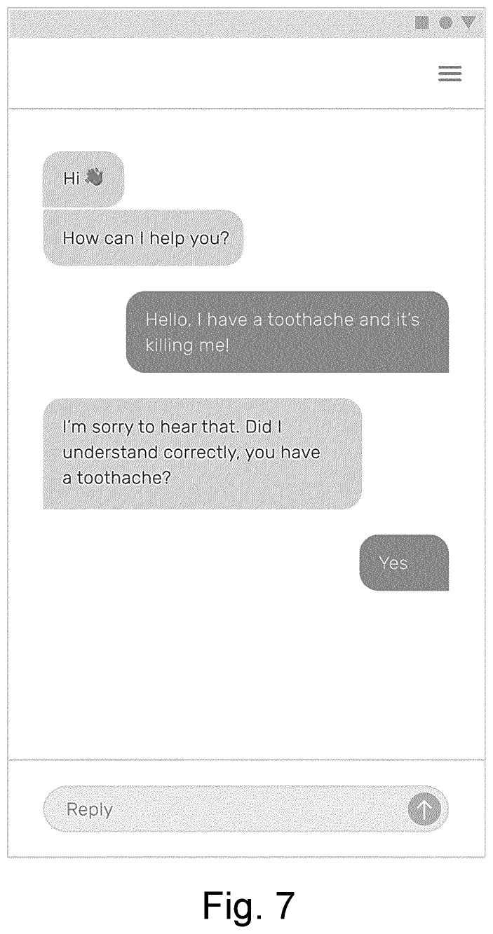

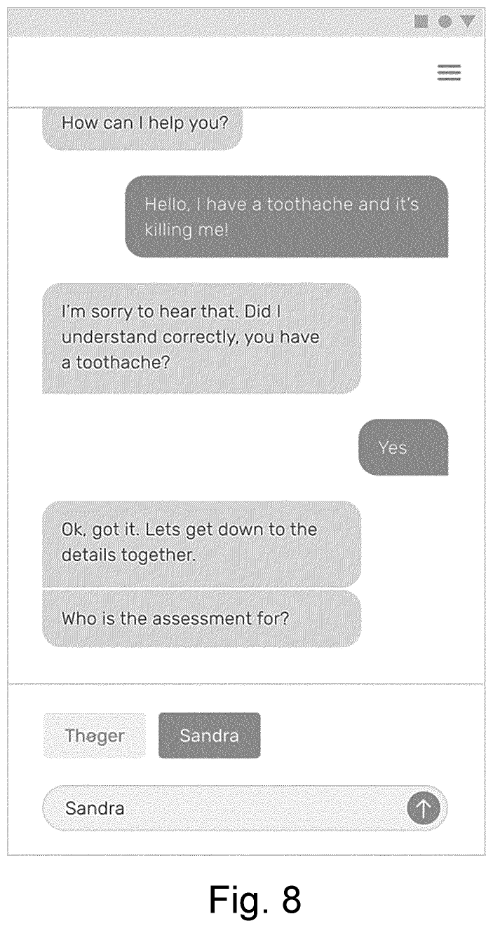

Initially, at step 511, the user 101 launches the app on the mobile device 103. The app is configured to perform a dialogue with the user 101, which dialogue comprises prompting the user 101 to indicate, for example, whether he/she wishes to continue an existing assessment, to view the results of a previous assessment, or to add a new assessment, as shown in FIG. 6. In another embodiment, the app is configured to require a login for use. The user 101 indicates “add new assessment” (FIG. 6) and may be prompted to indicate his oral health problem, e.g. toothache (FIG. 7). The app presents a list of selectable persons and/or may allow persons to be added, allowing the user 101 to select or identify the person whose oral cavity is to be captured on image, i.e. the person whom the oral health assessment is for (FIG. 8).

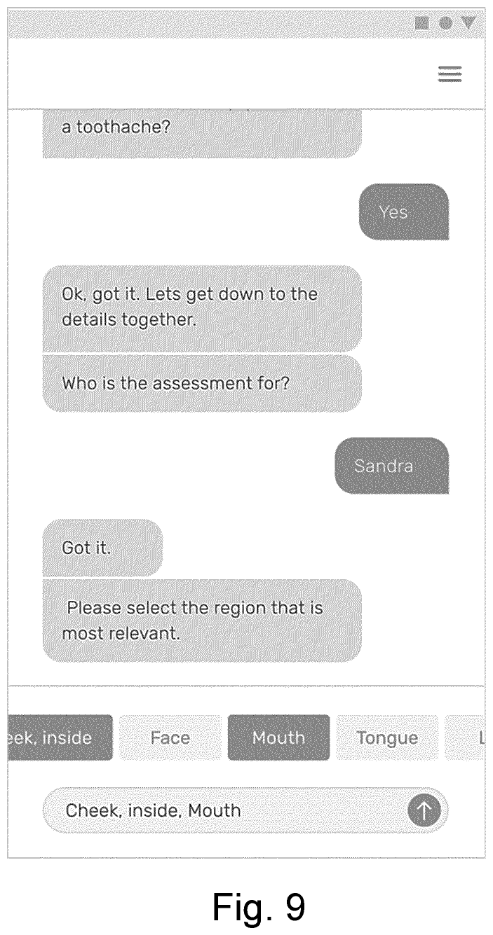

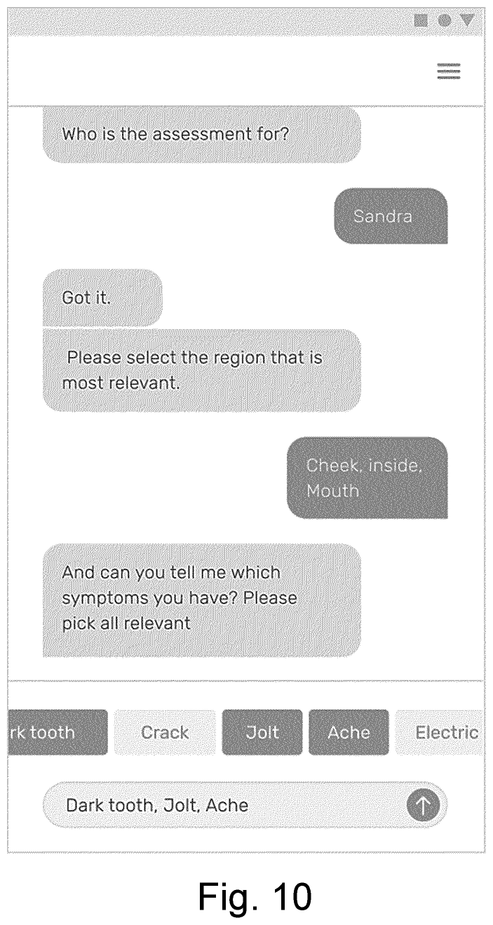

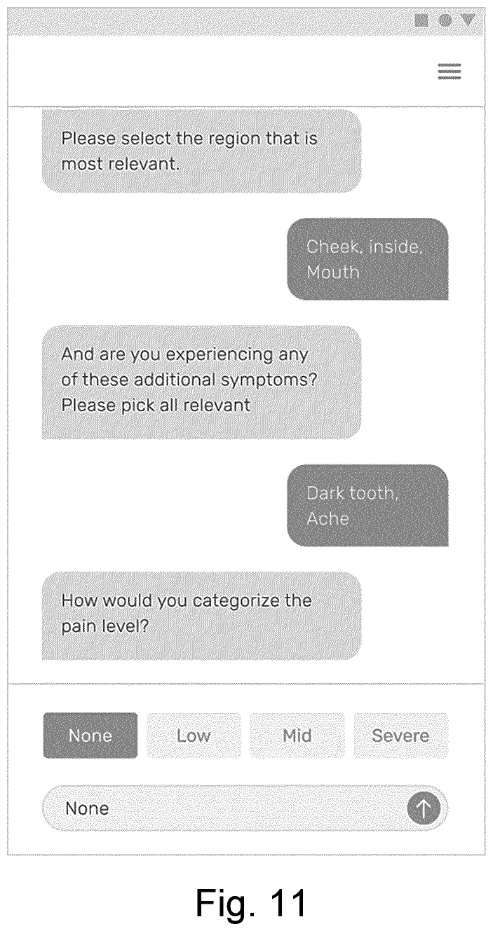

At steps 512 and 513, as part of the dialogue with the user, the app poses questions to the user 101 relating to the indicated oral health problem (if an oral health problem has been indicated), e.g. questions relating to pain, history of occurrence of symptoms, symptom triggers, and/or urgency, and receives the user's answers to the questions, which answers are recorded and processed locally on the mobile device and/or transmitted to a remote server 409 for processing. The questions are posed to the user 101 in the form of e.g. free text on the main screen 205, spoken text through a loudspeaker of the mobile device 103, a checklist on the main screen 205, a slider bar on the main screen 205, or a visual representation on the main screen 205. In an embodiment, a 3D representation of an oral cavity is presented to the user 101 for the user 101 to indicate in the representation an area corresponding to the area of the user's own oral cavity associated with the oral health problem. The area may e.g. be a specific tooth. The user's answers may be finite answers. For example, the user 101 may select one or more suggestions from a checklist (FIG. 9).

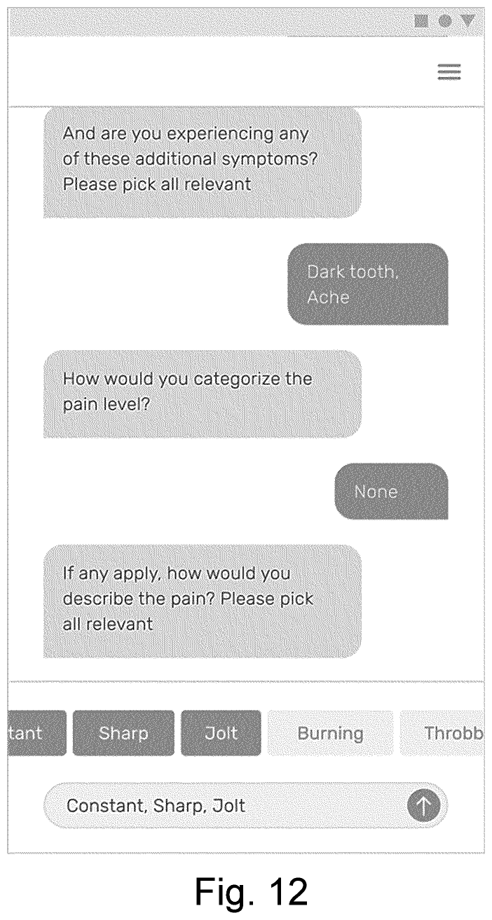

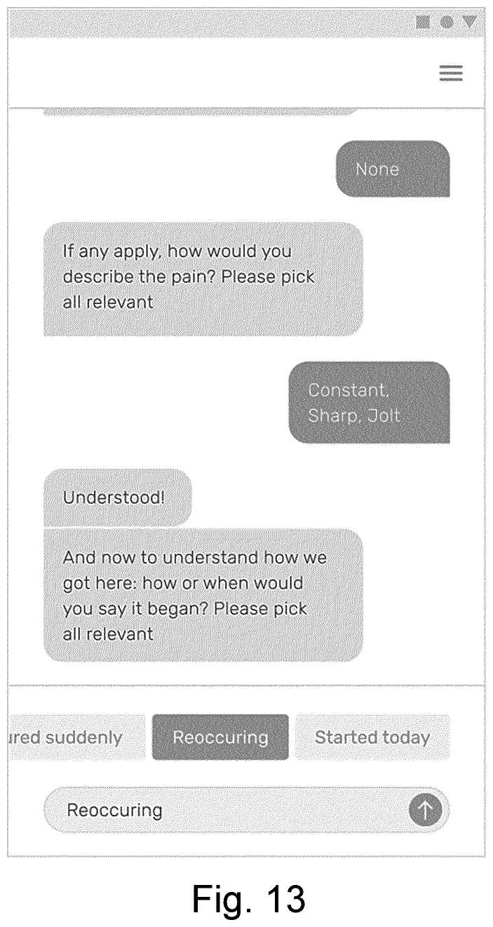

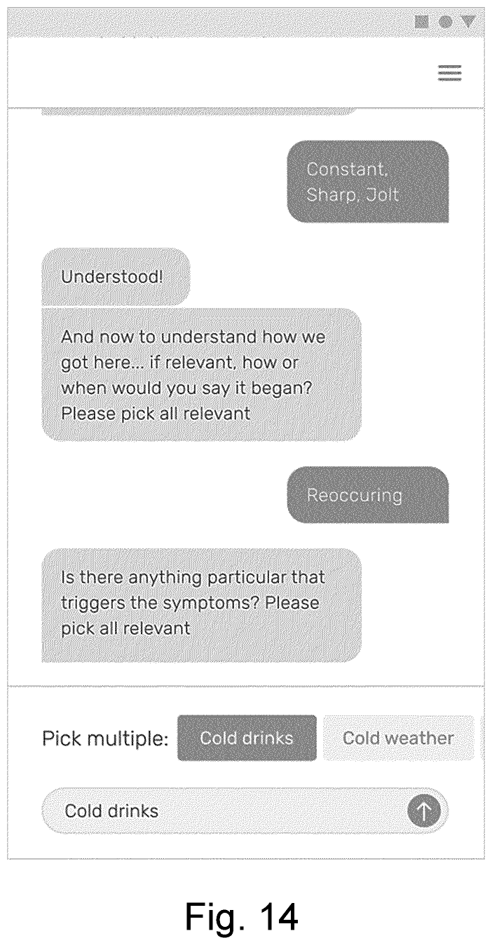

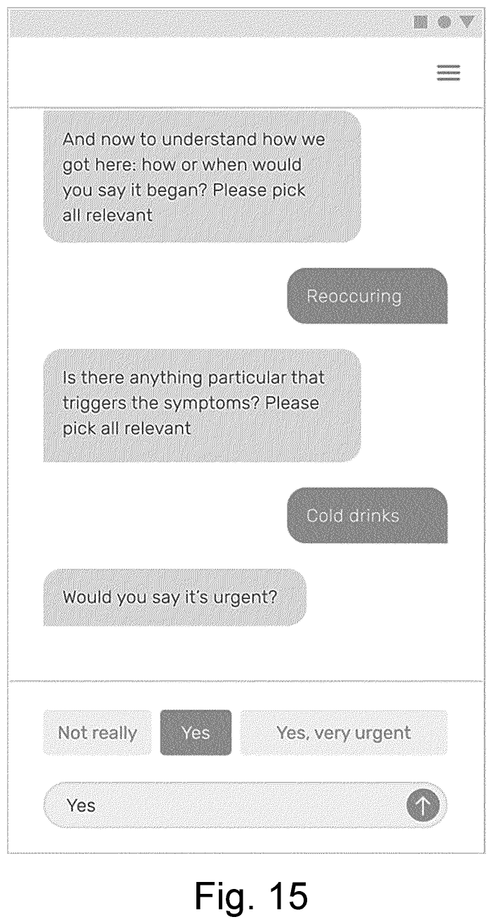

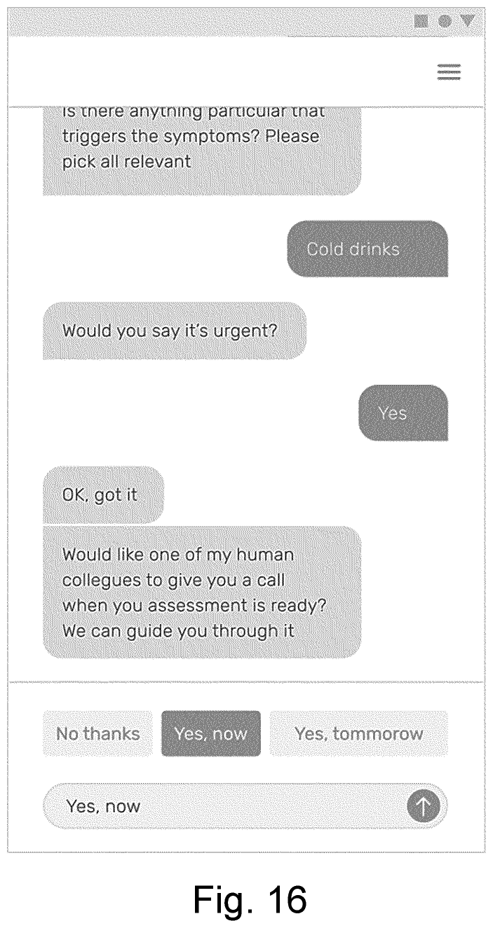

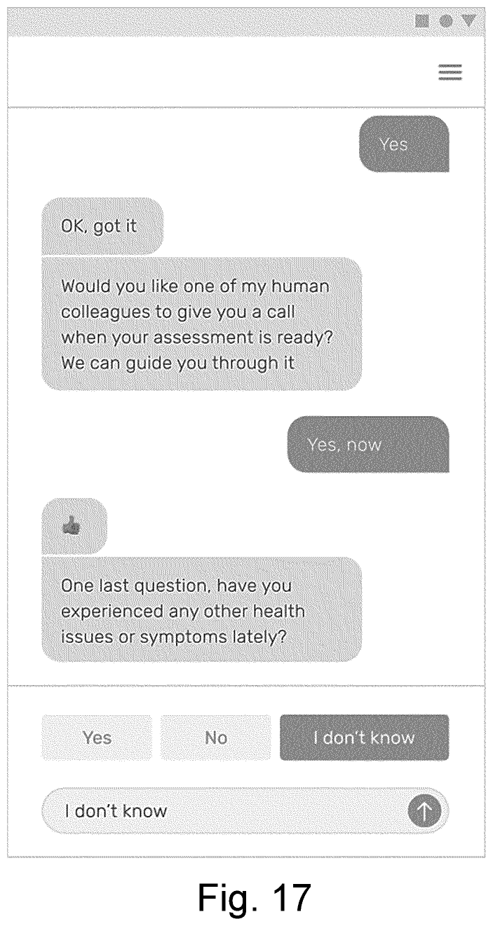

At step 512, the user 101 is prompted, for example, to localize a region/area associated with the oral health problem (FIG. 9), to describe symptoms (FIG. 10), to categorize the pain level (FIG. 11), to describe the pain (FIG. 12), the occurrence of the oral health problem (FIG. 13), symptom triggers (FIG. 14), the urgency (FIG. 15), to indicate whether he/she would like to be contacted by a service provider (e.g. application software hotline, insurance company, dental professional) (FIG. 16), and to indicate whether he/she has experienced any other health issues or symptoms lately (FIG. 17) (apart from the indicated oral health problem (FIG. 7)).

In an embodiment, the questions are preferably structured so as to lead to a result based on which an area of the oral cavity with symptoms can be determined with a finite number of questions and answers.

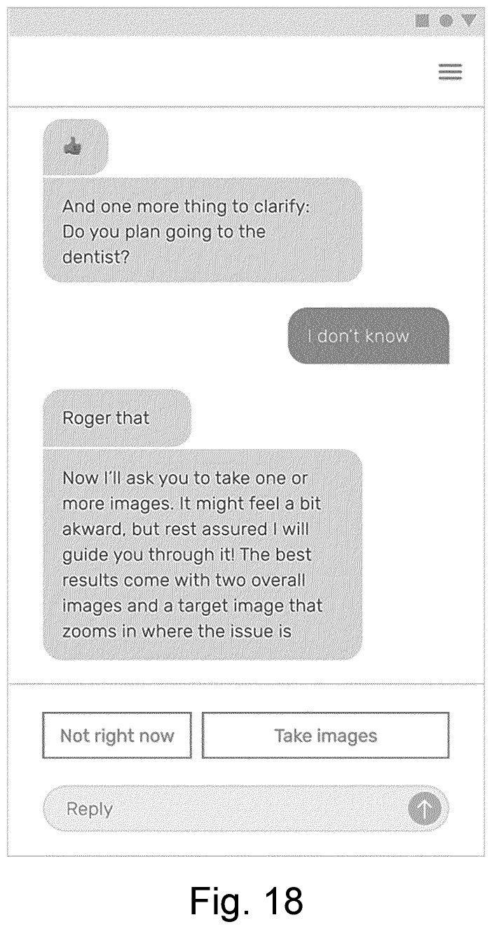

At step 514, the app prompts the user 101 to capture one or more images of an area of the user's oral cavity (FIG. 18), e.g. of an area with symptoms/an area associated with the oral health problem, which symptoms may, for example, be symptoms of at least one of gingivitis, periodontitis, dental caries, abrasion of tooth, bruxism, cold sore, erosion of teeth, fluorosis, herpes labialis, herpes zoster, or herpes infection. In an embodiment, based on the user's answers in step 512, an area of the user's oral cavity with symptoms is determined either by the software running on a processor of the mobile device 103 or remotely on a remote server, and the user is instructed by the app to capture an image of the determined area with symptoms.

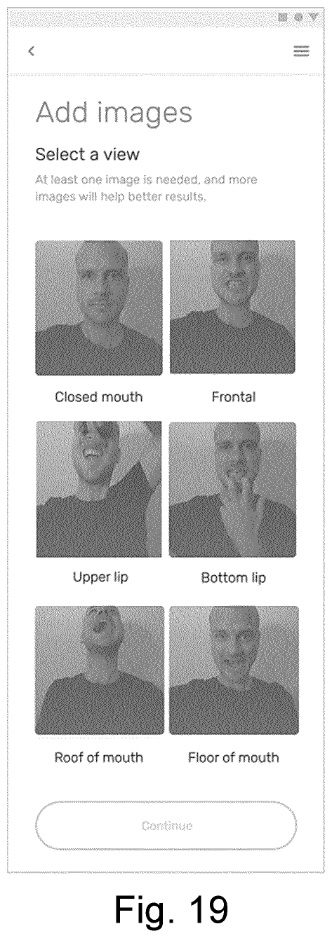

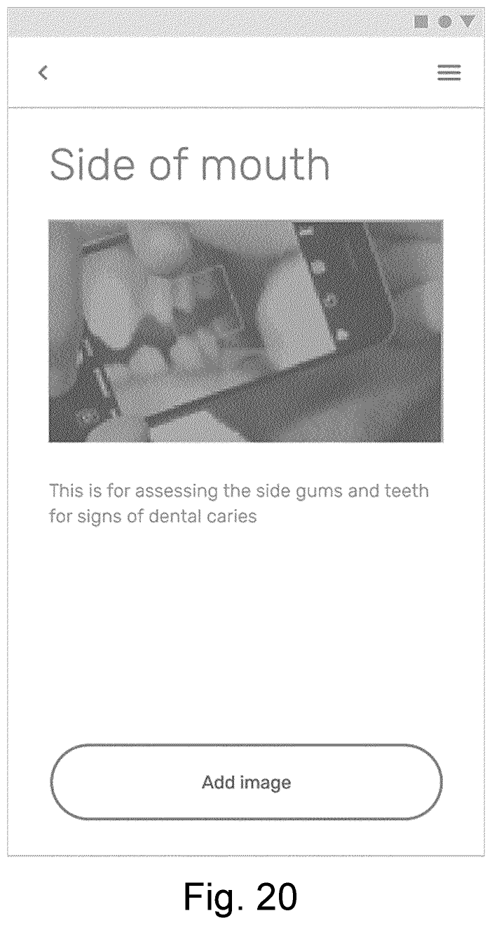

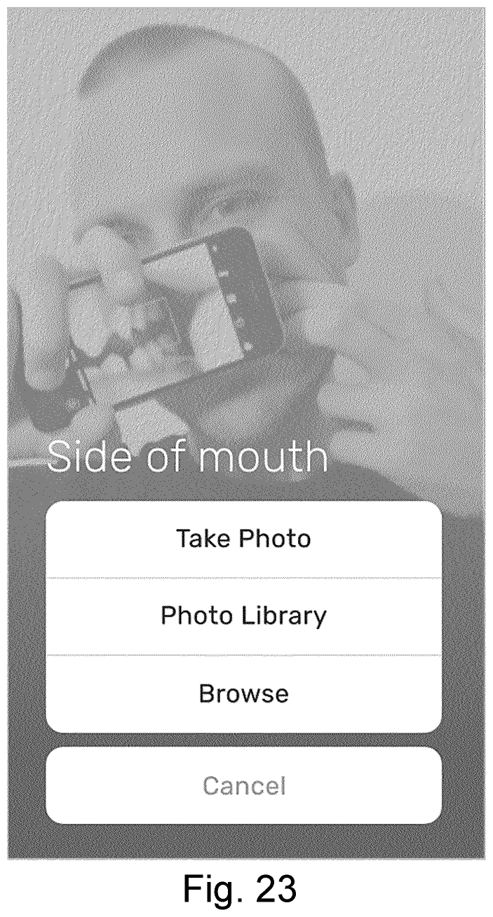

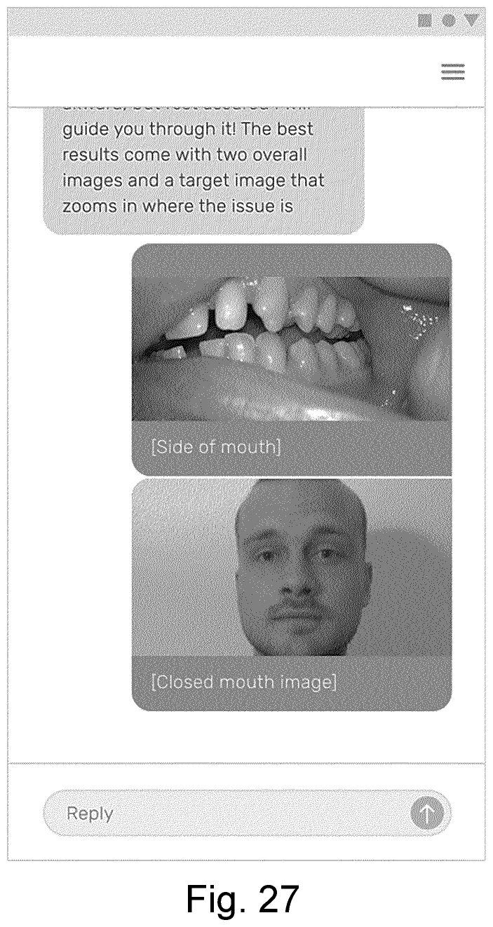

The user 101 may capture the images with the main camera 207 of the mobile device 103 and/or may add existing images from a gallery available e.g. on or via the mobile device 103 (FIG. 23). For capturing the images (or for adding existing images from a gallery), the user 101 is prompted to select a particular view/pose annotation (FIG. 19) describing the view/pose of the images, e.g. closed mouth view, bite view, bottom/lower arch view, upper arch view, bottom/lower lip view, upper lip view, closed bite anterior view, open bite anterior view, closed bite buccal view, open bite buccal view, roof of mouth view, floor of mouth view, side of mouth view, or frontal view, and is guided to capture an image in the selected particular view/pose (FIGS. 20, 21, 22). The captured images are annotated according to the selected view/pose (FIG. 27).

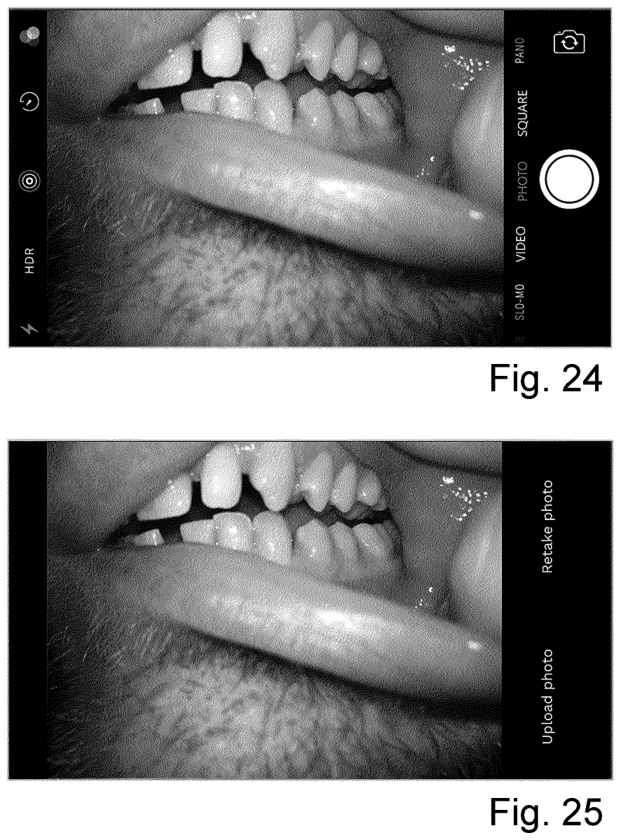

At steps 515 and 516, the user 101 is instructed to position him-/herself in front of a mirror 102 for capturing the one or more images of an area of his/her oral cavity and to rotate/flip the mobile device 103 such that the main screen 205 faces the mirror 102 and the main camera 207 faces the user 101 when capturing the images, with the main camera (207) being directed towards the area of the user's oral cavity. At step 517, the main camera 207 of the mobile device 103 captures one or more images of an area of the user's oral cavity (FIGS. 24, 25), e.g. of a portion of at least one of a tooth, a lip, or gums. For this, the app may be configured to control the main camera 207 of the mobile device 103. In an embodiment, prior to capturing the one or more images (step 517), the user 101 is instructed by the app to rotate the main camera (207). For example, the user may be instructed by the app to rotate the main camera (207) to landscape/horizontal position and to capture the one or more images of an area of his/her oral cavity in landscape mode.

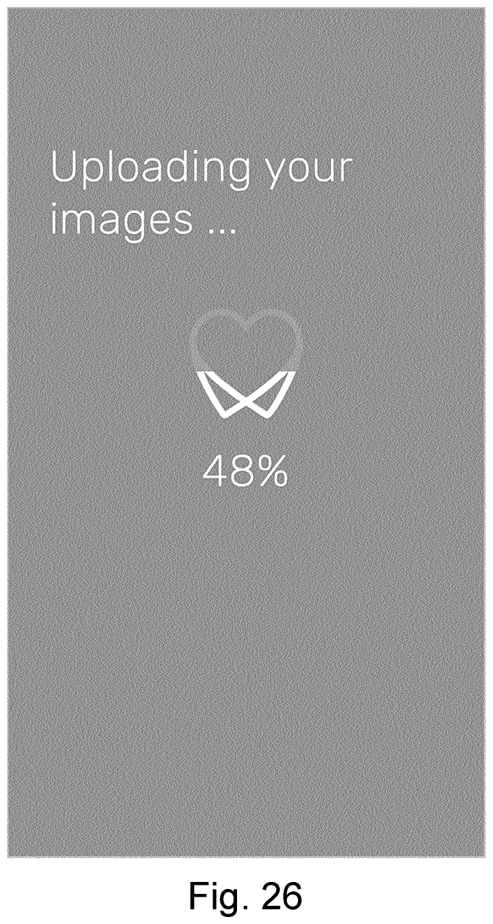

At step 518, the captured one or more image are saved on the mobile device 103 and processed locally on the mobile device 103 for assessment of the user's oral health based on the images. The images may be processed manually, automatically, or semi-automatically. In an embodiment, the processing and assessment is performed using artificial intelligence. Alternatively, or additionally, the captured images or representations thereof are transmitted (FIG. 26) to a remote server 409 for remote assessment of the user's oral health based on the captured images. The remote assessment is in an embodiment performed by a human expert, such as e.g. a dentist, and/or by software running on the remote server 409. The images may be used as input for statistical algorithms, machine learning, and/or deep learning technologies in order to assess the user's oral health. In an embodiment the software running on the remote server 409 uses artificial intelligence to assess the user's oral health based on the one or more images.

The assessment, whether performed locally or remotely, may comprise a referral to a dental professional, an estimate of the urgency of the oral health problem, a recommendation for self-treatment, etc. Particularly, the oral health assessment, performed locally and/or remotely, may comprise determining whether the user 101 is a candidate for an oral treatment, e.g. dental treatment or treatment of cold sores on the lips, based on the images and informing the user 101 on the mobile device 103, preferably via the app, whether he/she is a candidate for an oral treatment.

In an embodiment, as part of the processing of the one or more captured images, locally on the mobile device 103 or remotely on a remote server 409, the image quality of the one or more images or of a representation thereof is checked, preferably automatically. Particularly, it is checked whether the one or more images are of sufficient quality to allow for an assessment of the user's oral health based on the images, preferably whether the images have clinical image quality. The image quality check may comprise an assessment of e.g. focus, sharpness, lighting, resolution, etc. of the images. The user 101 is instructed by the app to capture another image of his/her oral cavity, if the image quality is below a threshold for image quality, i.e. if the images do not have clinical image quality.

Finally, at step 519, the user 101 receives a preliminary or final report comprising a preliminary assessment or final assessment of the user's oral health based on the one or more images of an area of the user's oral cavity.

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems.

The reference signs used in the claims shall not be construed as limiting the scope.

1-8. (canceled)

9. A computer program product encoded on a machine-readable non-transitory storage medium and operable to cause a processor of a mobile device (103) having a screen (205) on one side of a housing of the mobile device (103) and a camera (207) provided on the opposite side of the housing of the mobile device (103) to operate the mobile device to perform the steps of:
on the mobile device (103) performing, a dialogue with a user (101) of the mobile device (103),
the dialogue comprising prompting one or more questions to the user (101) via a user interface of the mobile device (103) and recording answers to the questions from the user (101) via the user interface of the mobile device (103),
determining an area of the user's oral cavity with symptoms based on the user's answers to the questions;
instructing the user (101) to position him-/herself in front of a mirror (102) for capturing an image of the area with symptoms,
instructing the user (101) to rotate the mobile device (103) such that the screen (205) faces the mirror (102) and the camera (207) faces the user (101), with the camera (207) being directed towards the area with symptoms for capturing an image of the area with symptoms,
capturing at least one image of the area with symptoms with the camera (207) of the mobile device (103) as instructed, and
either processing the at least one captured image locally on the mobile device (103) for assessment on the mobile device (103) of the user's oral health based on the at least one captured image, or
transmitting the at least one captured image or a representation thereof from the mobile device (103) to a remote server (409) for remote assessment of the user's oral health based on the at least one captured image.

10. (canceled)

11. The computer program product according to claim 9, comprising instructions for:
instructing the user (101) to rotate the camera (207) to landscape position and
capturing at least one image in landscape mode of the area of the user's oral cavity with the camera (207) of the mobile device (103).

12. The computer program product according to claim 9, comprising instructions for instructing the user (101) to position him-/herself such that the mirror (102) is at a given distance from the user's oral cavity.

13. The computer program product according to claim 9, comprising instructions for capturing with the camera (207) of the mobile device (103) an image of at least a portion of at least one of a tooth, a lip, or gums.

14. The computer program product according to claim 9, wherein the mobile device (103) is a smartphone or a tablet.

15. The computer program product according to claim 9, comprising instructions for displaying the image captured by the main camera (207) in real-time on the main screen (205) of the mobile device (103).

16. The computer program product according to claim 9, comprising instructions for instructing the user (101) to view the reflection of the area of the user's oral cavity displayed on the screen (205) of the mobile device (103) in the mirror (102) before/when capturing the image of the area of the user's oral cavity.

17. The computer program product according to claim 9, comprising instructions for guiding the user (101) to capture an image of the area of the user's oral cavity in a predefined view.
